(19) United States
(12) Patent Application Publication
(10) Pub. No.: US 2016/0106560 A1
(43) Pub. Date: Apr. 21, 2016

(54) VASCULAR IMPLANT FOR TREATING ANEURYSMS

(71) Applicant: Cerevatech Medical, Inc., San Jose, CA (US)

(72) Inventors: Kevin Kang, San Jose, CA (US); Jeffrey Madsen Pappas, San Jose, CA (US)

(21) Appl. No.: 14/853,586

(22) Filed: Sep. 14, 2015

Related U.S. Application Data
(60) Provisional application No. 62/179,706, filed on May 15, 2015.
(60) Provisional application No. 62/178,566, filed on Apr. 14, 2015.
(60) Provisional application No. 62/176,010, filed on Feb. 6, 2015.
(60) Provisional application No. 62/125,950, filed on Feb. 4, 2015.
(60) Provisional application No. 62/124,509, filed on Dec. 22, 2014.
(60) Provisional application No. 62/123,285, filed on Nov. 13, 2014.
(60) Provisional application No. 62/122,599, filed on Oct. 24, 2014.
(60) Provisional application No. 62/122,533, filed on Oct. 22, 2014.
(60) Provisional application No. 62/122,346, filed on Oct. 17, 2014.

(51) Int. Cl. A61F 2/88; A61F 2/89

(52) U.S. Cl. CPC A61F 2/885; A61F 2/89

(57) ABSTRACT

Implants and methods used for the treatment of aneurysms are disclosed. More particularly, embodiments of a vascular implant having several helical scaffolds, each helical scaffold including several undulations having short struts connected to long struts at an undulation joint, are disclosed. Other embodiments are also described and claimed.

This application claims the benefit of U.S. Provisional Patent Application No. 62/179,706 filed on May 15, 2015, U.S. Provisional Patent Application No. 62/785,566 filed on Apr. 14, 2015, U.S. Provisional Patent Application No. 62/176,010 filed on Feb. 6, 2015, U.S. Provisional Patent Application No. 62/125,950 filed on Feb. 4, 2015, U.S. Provisional Patent Application No. 62/124,509 filed on Dec. 22, 2014, U.S. Provisional Patent Application No. 62/123,285 filed on Nov. 13, 2014, U.S. Provisional Patent Application No. 62/122,599 filed on Oct. 24, 2014, U.S. Provisional Patent Application No. 62/122,533 filed on Oct. 22, 2014, and U.S. Provisional Patent Application No. 62/122,346 filed on Oct. 17, 2014, and this application hereby incorporates herein by reference those provisional patent applications.

### BACKGROUND

1. Field

The present invention relates to implants used for the treatment of vascular diseases. More particularly, embodiments of the present invention relate to a vascular implant having undulations arranged in a helical pattern around a longitudinal axis.

2. Background Information

Referring to FIG. 1, a pictorial view illustrating a patient with a cerebral aneurysm is shown. Aneurysms are pathological bulges in vascular anatomies, typically caused either by disease or weakening of a vessel wall. An aneurysm 100 may occur in the cerebral vessels 102 of a patient 104, such as in the vertebral, basilar, middle cerebral, posterior cerebral, or internal carotid arteries. Typically, cerebral vessels include vessel diameters in a range of between about 1.5 to 5.5 mm (3.5 mm average).

Referring to FIG. 2, a detail view, taken from Detail A of FIG. 1, of a neurovascular stent or a flow diversion device deployed at an aneurysm site is shown. Cerebral aneurysm 100 may be classified as a saccular aneurysm, having an aneurysm sac 202 joined with a portion of a vessel 102 at an aneurysm opening. Unless an aneurysm is depressurized, the aneurysm may eventually rupture, leading to severe complications. For example, in the case of cerebral aneurysms, a ruptured aneurysm may lead to severe intracranial hemorrhage with associated loss of perception, loss of balance, or even death.

Numerous approaches exist to treat cerebrovascular aneurysms, including some minimally invasive techniques. For example, an endovascular coiling procedure may be used in which a microcatheter is tracked to an aneurysm site and one or more embolic coils 204 are inserted into aneurysm sac 202 to promote blood clotting, which occludes and depressurizes the sac. Placing an implant 206 across the aneurysm opening may be used as an adjunct to, or a replacement for, embolic coil 204. For example, in a technique referred to as “jailing,” implant 206 may be a neurovascular stent delivered to scaffold the aneurysm opening and to create and/or retain a thrombus within the aneurysm sac. The aneurysm sac 202 may be occluded and depressurized by jailing embolic coil 204. Alternatively, implant 206 may be a flow diversion device that may be used without also deploying embolic coil 204. A flow diversion device may direct blood flow through vessel 102 and inhibit blood flow into the aneurysm sac 202 to depressurize aneurysm sac.

### SUMMARY OF THE DESCRIPTION

Vascular implants used for treating aneurysms are disclosed. In an embodiment, a vascular implant having an unexpanded state and an expanded state, e.g., a neurovascular stent or a flow diversion device, is provided. The vascular implant may include several helical scaffolds, e.g., three or more helical scaffolds, winding around a longitudinal axis from a first end to a second end of the vascular implant. Each helical scaffold may include a terminal undulation at the first end and several medial undulations helically offset from the terminal undulation between the first end and the second end. For example, the medial undulations may be arranged continuously in a sequence between the terminal undulation at the first end and a second terminal undulation at the second end. In an embodiment, each medial undulation includes a short strut connected to a long strut by a medial joint and the short strut is at least 20% shorter than the long strut. Furthermore, in an embodiment, a helical gap winds around the longitudinal axis between a first helical scaffold and a second helical scaffold, and a longitudinal distance across the helical gap is less than a difference in length between the short strut and the long strut.

Helical scaffolds of the vascular implant may extend along respective slant lines that spiral around the longitudinal axis. The slant lines may form a slant angle with a longitudinal split line that extends parallel to the longitudinal axis. For example, in an embodiment, the slant lines form a slant angle of less than 45 degrees with the longitudinal split line.

The terminal undulations at either end of the vascular implant may provide a transition between a first helical scaffold and a second helical scaffold. For example, the first helical scaffold may include a first terminal undulation having a first terminal strut connected to a second terminal strut by a first terminal joint, and the first terminal strut may extend across the helical gap to connect directly to the second helical scaffold. In an embodiment, the second terminal strut is also connected to the second helical scaffold, e.g., by a helix connector extending across the helical gap.

A connection scheme for undulations near the ends of vascular implant may vary, however. In an embodiment, vascular implant includes a second helical gap winding around the longitudinal axis between the second helical scaffold and a third helical scaffold. A first long strut of a first medial undulation helically adjacent to the first terminal undulation may be connected to the second helical scaffold. A second long strut of a second medial undulation helically adjacent to a second terminal undulation of the second helical scaffold, however, may not be connected to the third helical scaffold. Thus, connections between undulations near the ends of vascular implant may be chosen to balance flexibility and stability.

In addition to having different connector schemes, terminal undulations may include differing geometries. For example, the second terminal undulation may include a third terminal strut connected to a fourth terminal strut by a second terminal joint, and the second terminal joint may be circumferentially aligned with the first terminal joint. Although the terminal joints of adjacent terminal undulations may be aligned at one longitudinal end of the undulations, the other longitudinal end of the undulations may be misaligned. More particularly, a length of the first terminal strut may be different than a length of the third terminal strut. For example, the first terminal strut may be shorter than the third terminal strut, and thus, although a first end of the struts may be circumferentially aligned at a first end of the vascular implant, the other end of the first terminal strut may be farther from a second end of the vascular implant than the other end of third terminal strut (since the first terminal strut is shorter than the third terminal strut).

A connection scheme of vascular implant may also be chosen to achieve a desired torsional and/or longitudinal flexibility profile. For example, the first helical scaffold may include a helix length from the first end to the second end of vascular implant, and the first helical scaffold may be connected to the second helical scaffold by several helix connectors that extend across the helical gap. In an embodiment, the helix connectors are helically offset from each other by five or more medial undulations of the second helical scaffold over at least a majority of the helix length. The second helical scaffold may include a second helix length from the first end to the second end of vascular implant, and the second helical scaffold may be connected to a third helical scaffold by several second helix connectors that extend across a second helical gap. In an embodiment, the second helix connectors may be helically offset from each other by three or less medial undulations of the third helical scaffold over at least a majority of the second helix length. The five or more medial undulation separation between helix connectors may impart a relatively high flexibility between the first helical scaffold and the second helical scaffold. By contrast, the three or less medial undulations separation between helix connectors may impart a relatively low flexibility between the second helical scaffold and the third helical scaffold.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

FIG. 1 is a pictorial view illustrating a patient with a cerebral aneurysm.

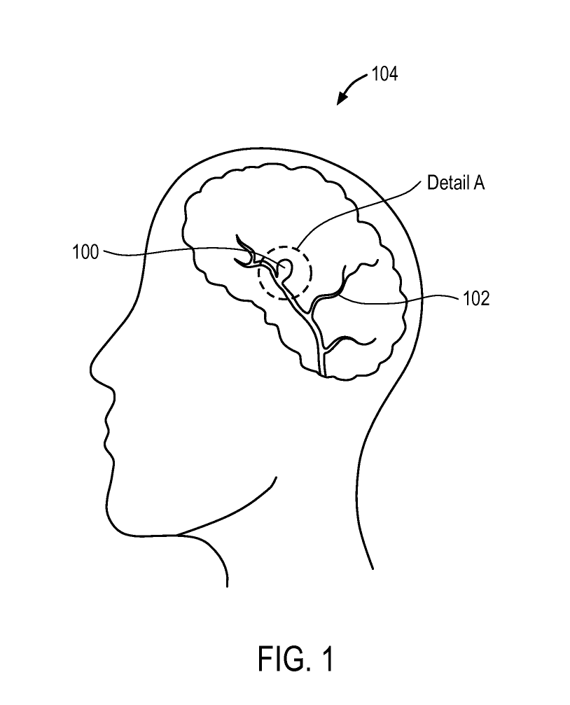

FIG. 2 is a detail view, taken from Detail A of FIG. 1, of an implant deployed at an aneurysm site.

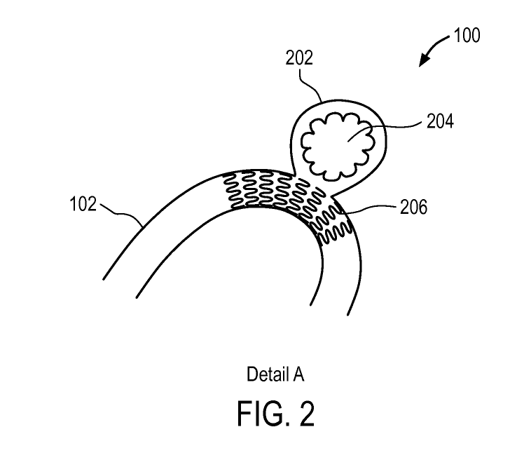

FIG. 3 is a plan view of a vascular implant pattern in accordance with an embodiment.

FIG. 4 is a detail view, taken from Detail B of FIG. 3, of a first end region of a vascular implant pattern in accordance with an embodiment.

FIG. 5 is a detail view, taken from Detail C of FIG. 4, of a medial undulation of a vascular implant pattern in accordance with an embodiment.

FIG. 6 is a plan view of a first terminal undulation region of a vascular implant pattern in accordance with an embodiment.

FIG. 7 is a plan view of a second terminal undulation region of a vascular implant pattern in accordance with an embodiment.

FIGS. 8A-8C are plan views of various helix connectors of a vascular implant pattern in accordance with an embodiment.

FIG. 9 is a plan view of a vascular implant pattern having a first helix connector scheme in accordance with an embodiment.

FIG. 10 is a plan view of a vascular implant pattern having a second helix connector scheme in accordance with an embodiment.

FIG. 11 is a plan view of a vascular implant pattern in accordance with an embodiment.

FIG. 12 is a plan view of a vascular implant pattern in accordance with an embodiment.

FIG. 13 is a pictorial view of an intravascular access path to an aneurysm site in a patient.

FIG. 14A-14B are pictorial views of various stages of deployment of a vascular implant at an aneurysm site in accordance with an embodiment of the invention.

### DETAILED DESCRIPTION OF THE INVENTION

While some embodiments of the present invention are described with specific regard to neurovascular applications, the embodiments of the invention are not so limited and certain embodiments may also be applicable to the treatment of aneurysms in other body vessels. For example, embodiments of the invention may be used to treat aneurysms distal to the origin of the renal arteries, thoracic aortic aneurysms, popliteal vessel aneurysms, or any other body vessel locations.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the present invention. In other instances, well-known processes and manufacturing techniques have not been described in particular detail in order to not unnecessarily obscure the present invention. Reference throughout this specification to “one embodiment,” “an embodiment,” or the like, means that a particular feature, structure, configuration, or characteristic described in connection with the embodiment is included in at least one embodiment of the invention. Thus, the appearances of the phrase “one embodiment,” “an embodiment,” or the like, in various places throughout this specification are not necessarily referring to the same embodiment of the invention. Furthermore, the particular features, structures, configurations, or characteristics may be combined in any suitable manner in one or more embodiments.

The use of relative terms may be used throughout the description, however, such terms are not intended to limit the use of a vascular implant to a specific configuration described in the various embodiments below. For example, a vascular implant may be described as having “first,” “second,” or “third” features, e.g., “a first terminal undulation” and “a second terminal undulation,” but the numerical indicators may be included to distinguish features shown in the accompanying figures and not to require that the features be placed in a particular order.

In an aspect, a vascular implant for treating aneurysms may include several helical scaffolds winding around a longitudinal axis. Each helical scaffold may include a series of undulations continuously arranged between ends of the vascular implant. More particularly, the undulations may include short struts connected to long struts such that adjacent helical scaffolds intermesh to increase a metal surface area within any given area of the vascular implant. Furthermore, the vascular implant pattern having helical scaffolds and intermeshed undulations may also minimize a number of undulation crowns or joints along any given transverse cross-section of the vascular implant. Thus, the profile of the vascular implant in an unexpanded state, e.g., a crimped state, may be minimized. For example, a vascular implant as described below may have a metal surface area of in the range of 11% to 22% and an outer diameter compatible with a 0.025-inch catheter in an unexpanded state.

In an aspect, a vascular implant includes several helical scaffolds interconnected by several helix connectors. Furthermore, the helix connectors may be helically offset relative to one another along a helical gap separating the helical scaffolds. A helix connector scheme, e.g., a number and spacing of helix connectors, may be chosen to achieve a desired torsional and longitudinal flexibility. For example, two or more of the helical scaffolds may be interconnected by helix connectors that are separated by five or more undulations to obtain a desired longitudinal and torsional flexibility. Similarly, two or more of the helical scaffolds may be interconnected by helix connectors that are separated by three or less undulations to obtain a desired longitudinal and torsional stiffness. Helix connector schemes may also be varied to enhance flexibility for better wall apposition and to enable retrievability of the vascular implant during delivery into a target anatomy. Several such helix connector schemes are described below.

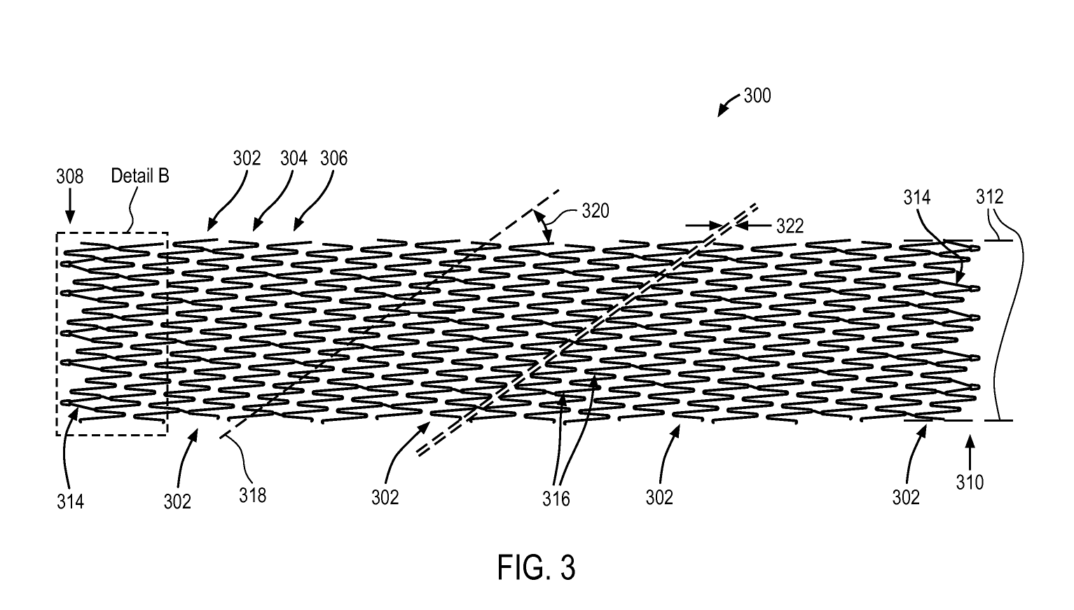

Referring to FIG. 3, a plan view of a vascular implant pattern is shown in accordance with an embodiment. Vascular implant 300 may be a medical implant such as a balloon expandable or self-expandable implant, which is useful for the treatment of vascular conditions. In particular, vascular implant 300 may be useful for the treatment of vascular aneurysms. For example, vascular implant 300 may be a neurovascular stent or a flow diversion device.

In an embodiment, vascular implant 300 includes several helical scaffolds winding around a longitudinal axis. For example, vascular implant 300 may include a first helical scaffold 302, a second helical scaffold 304, and a third helical scaffold 306 extending from a first end 308 to a second end 310 of vascular implant 300. As shown in FIG. 3, vascular implant 300 may include a total of five helical scaffolds, however, other embodiments of vascular implant 300 may include more or fewer helical scaffolds.

One skilled in the art will appreciate that the illustrated pattern of vascular implant 300 is a two dimensional representation of a three-dimensional object. More particularly, vascular implant 300 may be a tubular body, e.g., a scaffold formed by laser cutting a metallic tube, and the illustrated pattern may represent the scaffold after it is split along a longitudinal split line 312 and flattened. Split line 312 may be a reference line at a 0 degree and 360 degree position, i.e., split line 312 along the upper and lower edge of the flat pattern of vascular implant 300 may be the same reference line, which meets when the stent pattern is wrapped into a three-dimensional form. Accordingly, each helical scaffold is represented with several discontinuous diagonal segments spaced longitudinally between first end 308 and second end 310. For example, first helical scaffold 302 is illustrated as having five diagonal segments in the illustrated embodiment of vascular implant 300. One skilled in the art will appreciate that in three dimensions, the discontinuous diagonal segments of a respective helical scaffold would wrap around the tubular vascular implant 300 and join to form a continuous helical scaffold from end 308 to end 310.

Each helical scaffold includes a terminal undulation 314 at first end 308. Similarly, the helical scaffolds may include another terminal undulation 314 at second end 310 of vascular implant 300. Between the terminal undulations 314 of each helical scaffold, several medial undulations 316 may be helically offset from each other and from the terminal undulations 314. More particularly, each helical scaffold may include an elongated strut member that undulates from the first end 308 to the second end 310. As the elongated strut member undulates, it may follow the generally helical path between the terminal undulations 314 of the helical scaffold. The undulating path may include portions defined as terminal undulations 314 and portions defined as medial undulations 316. Thus, each helical scaffold may include a continuous arrangement of terminal undulations 314 and medial undulations 316.

The helical scaffolds spiral about the longitudinal axis, and thus, differ from conventional stent rings having stent cells arranged in a circumferential direction. More particularly, the helical scaffolds include undulations arranged along a slant line 318 that forms a slant angle 320 with split line 312. Slant angle 320 is an angle that is neither longitudinal, i.e., left-to-right in the figure, or circumferential, i.e., top-to-bottom in the figure. Slant angle 320 may be between 0 degrees to 90 degrees. In an embodiment, slant angle 320 is less than 45 degrees, as illustrated in each of the embodiments below. For example, slant angle 320 may range from 10 degrees to 35 degrees. In an embodiment, slant angle 320 is 14 degrees. In another embodiment, slant angle 320 is 32 degrees.

Slant angle 320 can be varied by varying the lengths of paired struts in the undulations such that each sequential undulation is helically offset from an adjacent undulation. Furthermore, slant angle 320 may be constant or may vary along the length of vascular implant 300. Accordingly, a pitch of the helical scaffolds may be constant or may vary along the length of vascular implant 300. Although slant line 318 is shown spiraling in a counterclockwise direction from first end 308 to second end 310 in FIG. 3, the helical scaffolds may be arranged to wind around the longitudinal axis along slant line 318 that spirals in a clockwise direction from first end 308 to second end 310.

In an embodiment, the helical scaffolds of vascular implant 300 extend along respective slant lines 318 having identical slant angles 320. Furthermore, each helical scaffold may wind around the longitudinal axis between two other helical scaffolds. More particularly, first helical scaffold 302 may be adjacent to and spaced apart from second helical scaffold 304, and second helical scaffold 304 may be adjacent to and spaced apart from third helical scaffold 306. The helical scaffolds may be separated from an adjacent helical scaffold by a respective helical gap 322. Thus, helical gaps 322 may wind around the longitudinal axis between helical scaffolds. Helical gap 322 may be defined as a void between adjacent helical scaffolds that is unoccupied by any undulations of the helical scaffolds. As described below, however, other structural features of vascular implant 300, such as helix connectors, may extend across helical gap 322 to connect undulations of adjacent helical scaffolds.

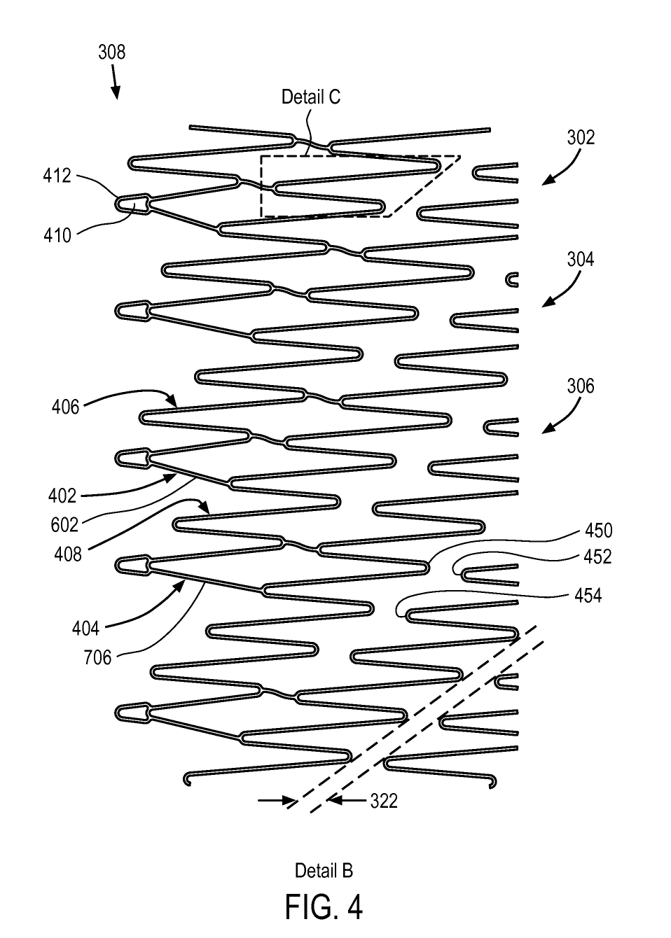

Referring to FIG. 4, a detail view, taken from Detail B of FIG. 3, of a first end region of a vascular implant pattern is shown in accordance with an embodiment. First helical scaffold 302, second helical scaffold 304, and third helical scaffold 306, terminate at first end 308 of vascular implant 300. The terminations may be at respective terminal undulations 314. For example, first helical scaffold 302 includes a first terminal undulation 402 and second helical scaffold 304 includes a second terminal undulation 404. Each terminal undulation 314 is immediately adjacent to an initial medial undulation 316 within the series of medial undulations 316 arranged helically from first end 308 to second end 310. For example, first terminal undulation 402 is immediately adjacent to a first medial undulation 406, and second terminal undulation 404 is immediately adjacent to a second medial undulation 408. As shown, all of the undulations may include pairs of struts connected to each other at a joint. In this description, undulations are considered to have strut pairs joined at a joint nearer to first end 308, and adjacent undulations are considered to be joined at joints nearer to second end 310 within any given helical scaffold. This convention is provided for the purpose of description, but such choice is not intended to be limiting.

The various joints of vascular implant 300 may also be used to define helical gap 322. For example, helical gap 322 may be a space between imaginary lines drawn through respective joints of adjacent helical scaffolds. As shown, a first imaginary line may be drawn through joints connecting adjacent undulations of a leftward helical scaffold. A second imaginary line may be drawn through joints connecting strut pairs of each undulation in an adjacent rightward helical scaffold. The space between the imaginary lines may form helical gap 322. As described below, distances across helical gap 322, e.g., longitudinally or circumferentially, may have predetermined relationships to other dimensions of vascular implant 300.

One or more terminal undulations 314 may be coupled with an end marker 410 to facilitate visualization of first end 308 under fluoroscopy. More particularly, a marker holder 412 may be located at a terminal undulation 314, e.g., coupled with a joint connecting struts of the terminal undulation 314. The marker holder 412 may be on a terminal undulation 314 at first end 308 or second end 310 of vascular implant 300. Each marker holder 412 may be loaded with a respective end marker 410. End marker 410 may include radiopaque material that can be stamped, injected, crimped, sputtered, or otherwise loaded into or onto marker holder 412. For example, the radiopaque material may be gold or another known radiopaque material. Marker holders 412 may have various shapes. For example, marker holder 412 is shown having a generally triangular shape, however, marker holder 412 may instead be shaped such that is includes a circular shape. Of course, other shapes such as other elliptical or polygonal shapes may be used for marker holder 412. Furthermore, some terminal undulations 314 may include marker holder 412 of a first shape, e.g., circular, and some terminal undulations 314 may include marker holder 412 of a different second shape, e.g., triangular, to help distinguish respective locations of the vascular implant. Thus, end marker 410 may be viewed under fluoroscopy to identify a position of first end 308 and/or second end 310 of vascular implant 300 and to facilitate accurate positioning of vascular implant 300 in vivo.

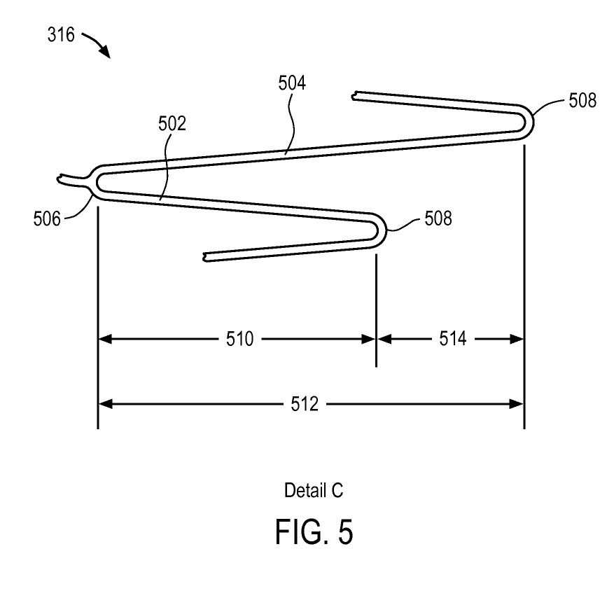

Referring to FIG. 5, a detail view, taken from Detail C of FIG. 4, of a medial undulation of a vascular implant pattern is shown in accordance with an embodiment. Medial undulations 316 may be serially arranged in a helical pattern to form intermediate scaffold lengths between terminal undulations 314 of respective helical scaffolds. Medial undulation 316 may include a short strut 502 and a long strut 504 connected by a medial joint 506. By way of definition, medial joint 506 may connect struts within a same medial undulation 316, and transition joint 508 may connect the medial undulation 316 to an adjacent medial undulation 316 within a same helical scaffold.

In an embodiment, each strut of medial undulation 316 may be a straight strut. That is, the struts may extend from medial joint 506 to transition joint 508 along a linear path. Alternatively, one or more of the struts may be curved, i.e., may follow a curvilinear path between medial joint 506 and transition joint 508. In either case, slant line 318 of the respective helical scaffold may be achieved through the different strut lengths of medial undulation 316. That is, since long strut 504 is longer than short strut 502, transition joint 508 connected to long strut 504 is longitudinally offset from transition joint 508 connected to short strut 502. By repeating this longitudinal offset between transition joints from undulation to undulation, a diagonal/helical undulation pattern is achieved.

In an embodiment, medial undulations 316 of the helical scaffold include short strut 502 having a short strut length 510 that is at least 20% shorter than a long strut length 512 of long strut 504. For example, long strut length 512 minus short strut length 510 may equal a length difference 514, and length difference 514 may be 25% to 50% of long strut length 512. By way of example, long strut length 512 may be in the range of 0.060 inches and 0.104 inches and short strut length 510 may be in the range of 0.044 inches and 0.076 inches, and thus, short strut 502 may be 27% shorter than long strut 504. Length difference 514 may directly correlate with slant angle 320. More particularly, when short strut length 510 and long strut length 512 are repeated in a series of undulations along a portion of a helical scaffold, a greater length difference 514 results in a smaller slant angle 320, and a lesser length difference 514 results in a greater slant angle 320. Thus, strut lengths may be chosen to result in a length difference 514 correlating with a desired slant angle 320.

In addition to slant angle 320 of a respective helical scaffold, strut lengths of medial undulations 316 may also be chosen to achieve a desired distribution of scaffold material. More particularly, length difference 514 may be chosen such that a medial joint 506 of one helical scaffold extends beyond a transition joint 508 of an adjacent helical scaffold. In other words, helical scaffolds may become intermeshed. As such, medial joints 506 and transition joints 508 of the helical scaffolds may be distributed such that a number of joints along any given transverse cross-section through vascular implant 300 is minimized.

In an embodiment, a longitudinal distance across helical gap 322 is less than length difference 514. For example, long strut 504 may have a long strut length 512 in a range of 0.060 inch to 0.104 inch, short strut 502 may have a short strut length 510 in a range of 0.044 to 0.076 inch, and helical gap 322 may have a longitudinal width between imaginary lines aligned with undulations joints of between 0.005 inches and 0.0125 inches. Thus, by way of example, length difference 514 may be—in the range of 0.016 inches and 0.028 inches whereas the longitudinal distance across helical gap 322 may be in the range of 0.005 inches and 0.0125 inches. Accordingly, referring again to FIG. 4, a reference transition joint 450 may be longitudinally leftward of a reference medial joint 452, and an adjacent medial joint 454 immediately adjacent to the medial undulation 316 having reference medial joint 452 may be longitudinally leftward of reference transition joint 450. Neither reference transition joint 450 nor reference medial joint 452 are circumferentially aligned with adjacent medial joint 454, and thus, when the pattern of vascular implant 300 is taken as a whole, undulation joints may be optimally distributed such that the number of joints on any transverse cross-section through vascular implant 300 is minimized. By way of example, there may be no more than 10 medial joints, e.g., there may be 7 medial joints along any transverse cross-section even though 16 medial joints are positioned around vascular implant 300 for each revolution of a given helical scaffold.

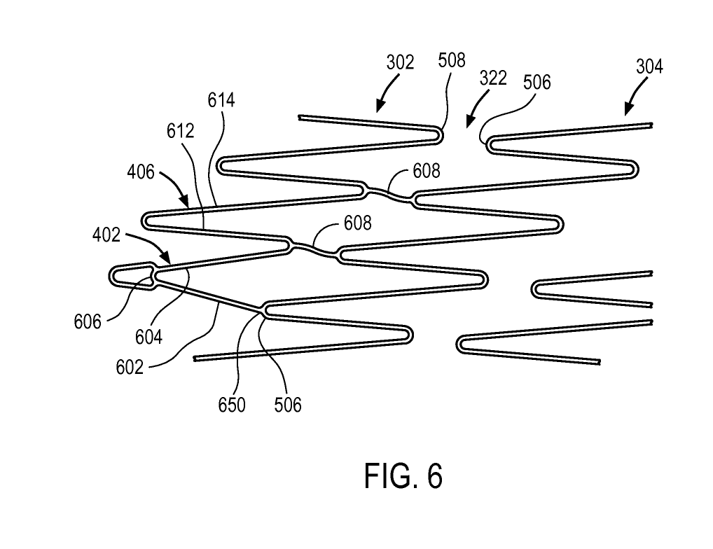

Referring to FIG. 6, a plan view of a first terminal undulation region of a vascular implant pattern is shown in accordance with an embodiment. To stabilize a helical scaffold at first end 308 and/or second end 310 of vascular implant 300, the helical scaffold includes a terminal undulation 314 that connects to a medial undulation 316 of an adjacent helical scaffold. First helical scaffold 302 may include first terminal undulation 402 having a first terminal strut 602 connected to a second terminal strut 604 by a first terminal joint 606. To stabilize first terminal undulation 402, e.g., to minimize relative movement between first helical scaffold 302 and second helical scaffold 304 when a longitudinal compressive load is applied to first terminal joint 606, one or more of first terminal strut 602 or second terminal strut 604 may be connected to respective medial joints 506 of second helical scaffold 304. For example, the first terminal strut 602 may connect directly to medial joint 506 of second helical scaffold 304. That is, first terminal strut 602 may extend from first terminal joint 606 across helical gap 322 so that an end of first terminal strut 602 connects directly to medial joint 506. In this way, helical scaffolds are terminated to respective adjacent helical scaffolds between first end 308 and second end 310 such that there is no exposed end of a helical scaffold, i.e., terminal joints are at the ends of vascular implant 300 rather than an end 650 of the elongated strut making up the helical scaffold. In an embodiment, first terminal strut 602 is straight, and it is contemplated that the straight strut may reduce an unexpanded state profile of vascular implant 300 as compared to first terminal strut 602 having a curved shape.

First terminal strut 602 may connect to a medial joint 506 that is longitudinally aligned with first terminal joint 606, or is circumferentially offset from first terminal joint 606. For example, first terminal strut 602 is shown slanting down and to the right from first terminal joint 606 to medial joint 506, which is circumferentially offset from first terminal joint 606. First terminal strut 602 may, however, slant up and to the right (or straight to the right) between first terminal joint 606 and a different medial joint 506, or the same medial joint 506 in a different location.

Second terminal strut 604 may also be connected to second helical scaffold 304. In an embodiment, a helix connector 608 may extend across helical gap 322 from a transition joint 508 (attached to second terminal strut 604) to an adjacent medial joint 506 of second helical scaffold 304. Helix connector 608 may have numerous shapes, as described below, and may be sized to longitudinally offset the transition joint 508 connected to second terminal strut 604 from the medial joint 506 of second helical scaffold 304. Furthermore, helix connector 608 may provide column strength to resist relative movement between the transition joint 508 and the medial joint 506 when a longitudinal compressive load is applied to first terminal joint 606. Thus, first terminal undulation 402 may include terminal struts connected to an adjacent helical scaffold directly, or indirectly by helix connector 608. In an embodiment, all terminal undulations 314 of vascular implant 300 have both terminal struts connected to an adjacent helical scaffold in the same manner, e.g., one strut connected directly and another strut connected indirectly.

In an embodiment, a medial undulation 316 immediately adjacent to a terminal undulation 314 may also include connections between both undulation struts and an adjacent helical scaffold. For example, first medial undulation 406 may include a first short strut 612 connected to second terminal strut 604 by a transition joint 508, and thus, may be helically offset from first terminal undulation 402 within first helical scaffold 302. Furthermore, first short strut 612 may be connected to a first long strut 614 of first medial undulation 406. As described above, a helix connector 608 may connect to the transition joint 508 that joins first short strut 612 with second terminal strut 604, and the helix connector 608 may extend across helical gap 322 to second helical scaffold 304. In an embodiment, first long strut 614 is similarly connected to second helical scaffold 304. More particularly, a helix connector 608 may extend from first long strut 614 (or a transition joint 508 connected to first long strut 614) across helical gap 322 to a medial joint 506 of second helical scaffold 304. The connection between first long strut 614 and second helical scaffold 304 may be used to limit relative movement between first medial undulation 406 and second helical scaffold 304 when a longitudinal compressive load is applied to first end 308. Such connections may be incorporated in the pattern of vascular implant 300 to maintain the longitudinal compressibility of the implant below a predetermined threshold, however, such connections need not be incorporated along every helical scaffold.

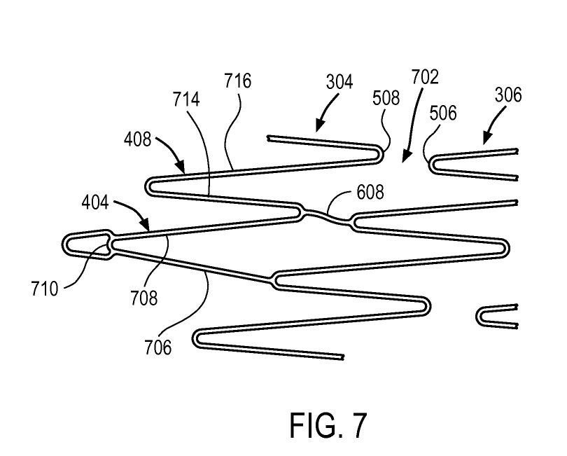

Referring to FIG. 7, a plan view of a second terminal undulation region of a vascular implant pattern is shown in accordance with an embodiment. A second helical gap 702 may wind around the longitudinal axis between second helical scaffold 304 and third helical scaffold 306. In certain respects, a termination scheme of second helical scaffold 304 may be similar to that described above with respect to first helical scaffold 302 in FIG. 6. For example, second helical scaffold 304 may include a second terminal undulation 404 having a third terminal strut 706 connected to a fourth terminal strut 708 by a second terminal joint 710. Furthermore, third terminal strut 706 may extend across second helical gap 702 to connect directly to a medial undulation 316 of third helical scaffold 306, while fourth terminal strut 708 may be connected to the third helical scaffold 306 by a helix connector 608 that extends across second helical gap 702 to another medial undulation 316. The geometries of first terminal undulation 402 and second terminal undulation 404, however, may not be identical.

In an embodiment, third terminal strut 706 that connects directly to third helical scaffold 306 by extending from second terminal joint 710 across second helical gap 702 has a length that is different than a length of first terminal strut 602. More particularly, the straight struts of each terminal undulation 314 that connect directly to adjacent helical scaffolds may be sized to maintain respective terminal joints at a common longitudinal position. By way of example, with reference to FIG. 4, it can be seen that third terminal strut 706 of second terminal undulation 404 is longer than first terminal strut 602 of first terminal undulation 402 and as a result first terminal joint 606 and second terminal joint 710 are circumferentially aligned, i.e., aligned vertically in the illustrated flat pattern. As such, end markers 410 coupled with respective terminal joints may also be circumferentially aligned around vascular implant 300.

Referring again to FIG. 7, an additional variation in helical scaffolds may be the connection scheme of medial undulations immediately adjacent to terminal undulations. For example, second helical scaffold 304 may include a second medial undulation 408 similar to first medial undulation 406. That is, second medial undulation 408 may also include a second short strut 714 connected to a second long strut 716. Second short strut 714 may be helically adjacent to fourth terminal strut 708, and thus, may be connected to the third helical scaffold 306 by the same helix connector 608. In contrast to first long strut 614 of first medial undulation 406, however, second long strut 716 of second medial undulation 408 may not connect to third helical scaffold 306 either directly or by way of a helix connector 608. More particularly, a transition joint 508 attached to second long strut 716 may be disconnected from a medial joint 506 across second helical gap 702, and thus, second helical scaffold 304 is not connected to third helical scaffold 306 between the transition joint 508 and the corresponding medial joint 506.

As described above, helix connectors 608 may extend across helical gaps between adjacent helical scaffolds to connect the helical scaffolds and maintain structural integrity of vascular implant 300. More particularly, helix connectors 608 may be positioned such that vascular implant 300 has a desired torsional and longitudinal flexibility. Helix connectors 608 of vascular implant 300 may have a same or different geometry, and the geometry may be varied in different embodiments of vascular implant 300. For example, the geometry may vary based on a waviness and a location of the connected joints.

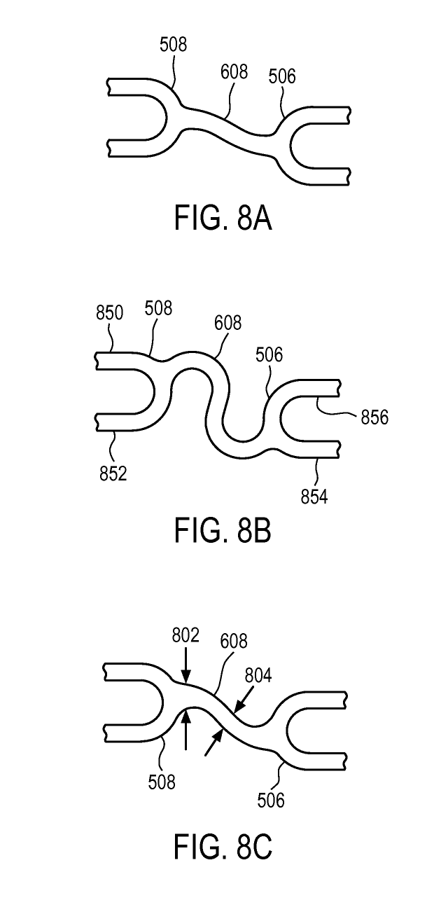

Referring to FIG. 8A, a plan view of a helix connector of a vascular implant pattern is shown in accordance with an embodiment. Helix connector 608 may extend between transition joint 508 and medial joint 506 across a helical gap in a straight configuration. In an embodiment, helix connector 608 extends from a middle of transition joint 508 to a middle of medial joint 506, and transition and medial joint 506 are longitudinally aligned.

Referring to FIG. 8B, a plan view of a helix connector of a vascular implant pattern is shown in accordance with an embodiment. Helix connector 608 may have an “S” curve geometry. More particularly, helix connector 608 may follow a curvilinear path having at least two radii between the joints. Helix connector 608 may extend from transition joint 508 nearer to one undulation strut 850 than another undulation strut 852. Similarly, helix connector 608 may connect to medial joint 506 nearer to one undulation strut 854 than another undulation strut 856. Accordingly, helix connector 608 may provide rotational flexibility to minimize unintentional twisting.

Referring to FIG. 8C, a plan view of a helix connector of a vascular implant pattern is shown in accordance with an embodiment. Helix connector 608 may have a width that varies across a helical gap. For example, a connection width 802 of helix connector 608 near a connection point with an undulation joint 506 or 508 may be greater than a middle width 804 of helix connector 608 near the middle of the helical gap.

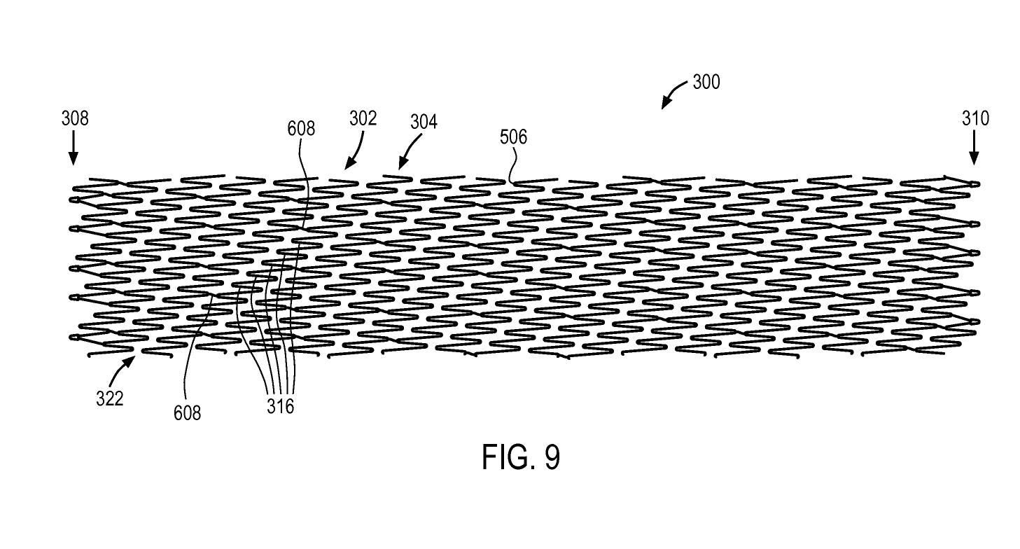

Referring to FIG. 9, a plan view of a vascular implant pattern having a first helix connector scheme is shown in accordance with an embodiment. In an embodiment, first helical scaffold 302 includes a helix length from first end 308 to second end 310 of vascular implant 300. Similarly, second helical scaffold 304 includes a second helix length from first end 308 to second end 310. First helical scaffold 302 may be connected to second helical scaffold 304 along helix length by several helix connectors 608, which extend across helical gap 322 between respective joints of the helical scaffolds. The position of helix connectors 608 may be varied to adjust the number and locations of connections between adjacent helical scaffolds, and thus, to control the torsional and longitudinal flexibility of vascular implant 300.

Helix connectors 608 may be positioned to impart a relatively high flexibility to vascular implant 300. For example, helix connectors 608 may be helically offset from each other along helical gap 322 such that five or more medial undulations 316 of second helical scaffold 304 are between the pair of helix connectors 608. That is, five or more medial joints 506 of the intermediate medial undulations 316 may be between the pair of helix connectors 608. The helix connector scheme may be repeated over at least a majority of the helix length. For example, every pair of helix connectors 608 connecting medial undulations 316 of first helical scaffold 302 and medial undulations 316 of second helical scaffold 304 along helical gap 322 between first end 308 and second end 310 of vascular implant 300 may be separated by five or more medial undulations 316. It will be appreciated that, while helix connectors 608 in FIG. 9 are shown as being separated by five medial undulations 316, separating helix connectors 608 by six medial undulations 316 may provide for a more flexible vascular implant 300. Similarly, adjacent helical scaffolds that are connected by helix connector 608 pairs separated by fewer than five medial undulations 316, e.g., by four or fewer medial undulations 316, may provide for a less flexible vascular implant 300.

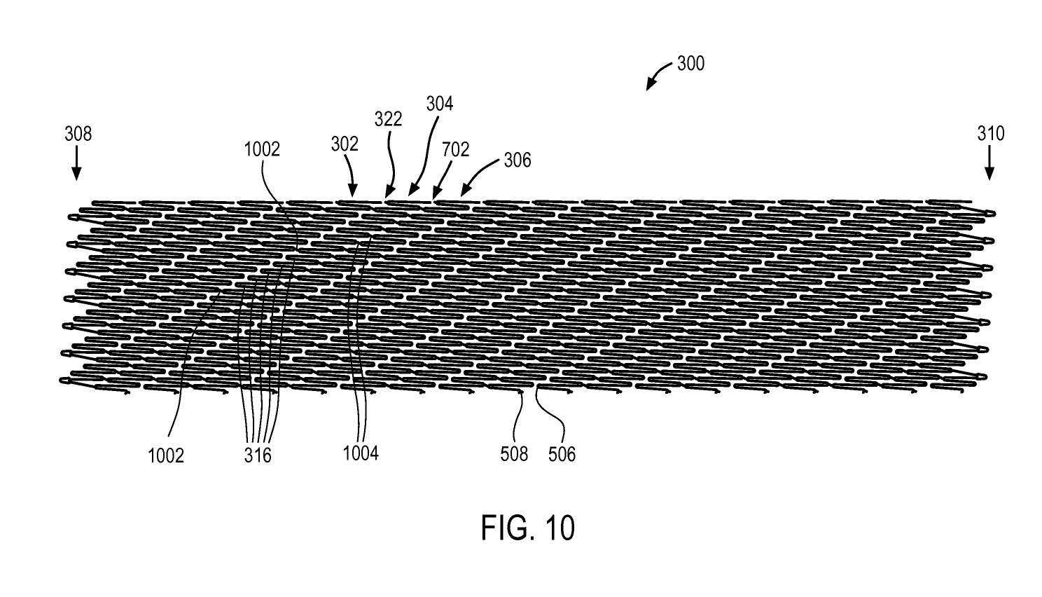

Referring to FIG. 10, a plan view of a vascular implant pattern having a second helix connector scheme is shown in accordance with an embodiment. In an embodiment, different pairs of helical scaffolds are interconnected by helix connectors 608 having different connector schemes. For example, first helical scaffold 302 and second helical scaffold 304 may be interconnected by first helix connectors 1002 having a connector scheme such as the scheme described above with respect to FIG. 9. That is, five or more medial joints 506 of the intermediate medial undulations 316 may be between the pair of helix connectors 1002. By contrast, second helical scaffold 304 may be interconnected with third helical scaffold 306 by second helix connectors 1004 having a different connector scheme. For example, second helix connectors 1004 extending across second helical gap 702 between second helical scaffold 304 and third helical scaffold 306 may be helically offset from each other by three or less medial undulations 316. That is, three or fewer medial joints 506 of the intermediate medial undulations 316 may be between the pair of helix connectors 1004. The connector pattern may be repeated over at least a majority of the second helix length, and thus, connections between second helical scaffold 304 and third helical scaffold 306 may impart a higher cumulative stiffness to vascular implant 300 than the connections between first helical scaffold 302 and second helical scaffold 304. In an embodiment, helix connectors 608 between helical scaffolds may not be separated by medial undulations 316. For example, as shown in FIG. 10, the second helix connector scheme may include a second helix connector 1004 between every transition joint 508 of second helical scaffold 304 and every medial joint 506 of third helical scaffold 306. In addition to altering the flexibility of vascular implant 300, the second helix connector scheme may provide a retrievable vascular implant 300. More particularly, since every other pair of helical scaffolds is connected at every joint in FIG. 10, vascular implant 300 may be deployed from a delivery sheath by retracting the delivery sheath and then captured back into the delivery sheath by advancing the delivery sheath.

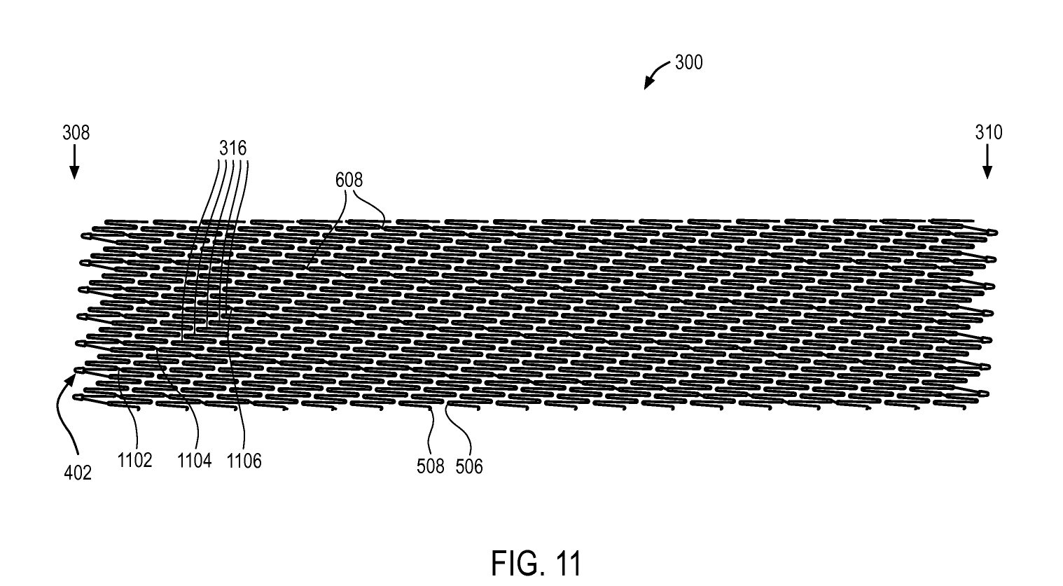

Referring to FIG. 11, a plan view of a vascular implant pattern is shown in accordance with an embodiment. Vascular implant 300 pattern having the features described above may be varied to create vascular implant 300 for specific neurovascular applications. For example, vascular implant 300 described above with respect to, e.g., FIGS. 3 and 9, may be particularly useful as a neurovascular stent. The pattern of vascular implant 300, however, may be altered to create vascular implant 300 that is particularly useful as a flow diversion device. One skilled in the art would understand how to alter the features to achieve this application-specific design. As an example of such an alteration, vascular implant 300 may include helical gaps having a longitudinal gap distance of between 0.005 inch to 0.006 inch to create a more dense pattern with a higher metal surface area as compared to patterns of FIGS. 3 and 9. As a result, vascular implant 300 having a flow diversion design may divert blood flow away from an aneurysm more effectively than the neurovascular stent designs discussed above.

Another pattern variation shown in FIG. 11 includes a first helically offset helix connector that is helically offset from a terminal undulation by a shorter distance than a next helically offset helix connector. More particularly, first terminal undulation 402 may include a reference helix connector 1102 connected to a medial joint of an adjacent helical scaffold. This connection scheme is similar to that described above. A first offset helix connector 1104 may be helically offset from reference helix connector 1102 by at least one medial undulation 316 of the adjacent helical scaffold. That is, at least one medial joint 506 of the intermediate medial undulation 316 may be between the pair of helix connectors 1102, 1104. Furthermore, the helical offset between reference helix connector 1102 and first offset helix connector 1104 may be less than a helical offset between first offset helix connector 1104 and a second offset helix connector 1106. As shown, reference helix connector 1102 and first offset helix connector 1104 may be separated by two medial undulations of the adjacent helical scaffold. By contrast, first offset helix connector 1104 may be separated from second offset helix connector 1106 by five medial undulations 316 of the adjacent helical scaffold. Furthermore, one or more helix connectors 608 between second offset helix connector 1106 and second end 310 of vascular implant 300 may be helically offset from each other by a same number of medial undulations 316. For example, several pairs of helix connectors 608 are helically offset by five medial undulations 316 of the adjacent helical scaffold between second offset helix connector 1106 and second end 310. As described above, the non-uniform offsets between helix connectors 608 of vascular implant 300, e.g., reference helix connector 1102, first offset helix connector 1104, and second offset helix connector 1106, may be placed to produce uniform structural flexibility while reducing a likelihood of non-uniform axial displacement under a longitudinal compressive load.

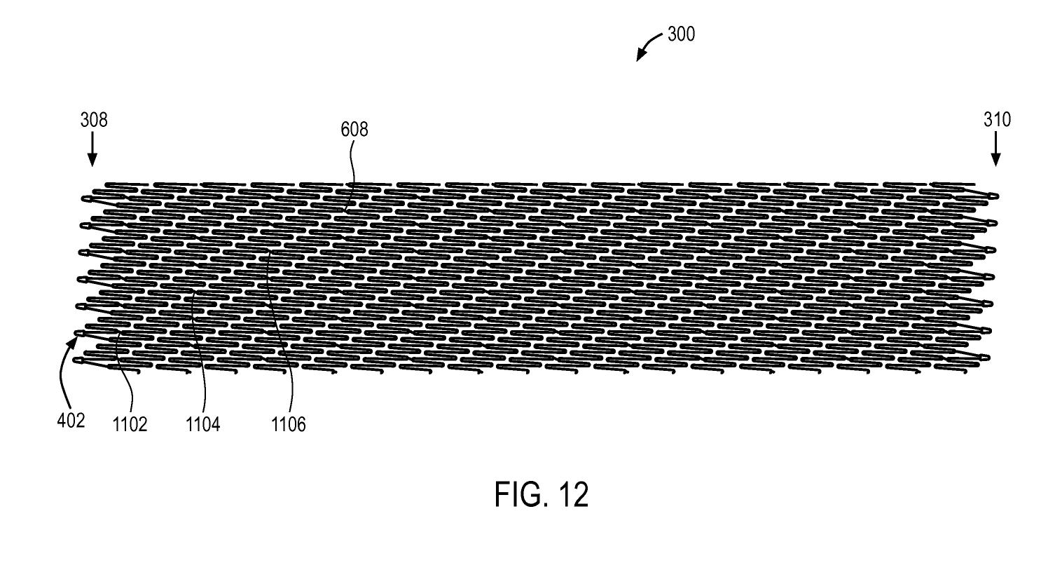

Referring to FIG. 12, a plan view of a vascular implant pattern is shown in accordance with an embodiment. It will be appreciated that the non-uniform connector scheme of the vascular implant 300 pattern shown in FIG. 11 is similar to that shown in FIG. 3 above. Thus, flow diverter designs and neurovascular stent designs for vascular implant 300 may include at least some features that are identical or similar. In FIG. 12, it can be seen that reference helix connector 1102, which connects first terminal undulation 402 to an adjacent helical scaffold, may be offset from first offset helix connector 1104 by the same number of medial undulations 316 by which first offset helix connector 1104 is helically offset from second offset helix connector 1106. This helix connector scheme is similar to that shown in FIG. 9 above. The embodiment further illustrates that vascular implant 300 pattern may be altered for specific neurovascular applications, e.g., stenting or flow diversion, while leveraging many of the same pattern features across designs. Furthermore, it will be appreciated that these alterations may require trade-offs between designs. For example, non-uniform connector schemes such as those shown in FIGS. 3 and 11 may advantageously distribute loads in a manner that maintains a circular vessel lumen at a cost of inducing higher localized stresses in struts. By contrast, uniform connector schemes such as those shown in FIGS. 9 and 12 may advantageously distribute stresses to improve fatigue life at a cost of causing pancaking and flattening of vascular implant 300 when deployed in a tortuous target vessel. Accordingly, features of the patterns described above may be combined and/or modified to create vascular implant 300 that is effective for a variety of use cases.

One skilled in the art would understand that the pattern of vascular implant 300 may be varied in numerous other manners to achieve a desired performance. For example, a strut width of undulation struts (terminal undulations or medial undulations) may be varied to affect radial strength response of vascular implant 300. In an embodiment, the strut width may be between 0.0010 to 0.0012 inches, although other strut widths may be used. Furthermore, the strut width in combination with the dense pattern enabled by the helical scaffolds described above may allow vascular implant 300 to have a metal surface area that would be considered high by one skilled in the art. For example, vascular implant 300 may exhibit a metal surface area in a range between 11% to 22% in the exemplary patterns illustrated by the accompanying figures. Of course, the metal surface area depends on the expansion state of vascular implant 300. For example, the metal surface area of vascular implant 300 may be 22% in the unexpanded state and decrease to 11% in the expanded state.

Vascular implant 300 may be fabricated using manufacturing processes that are known in the field of stent manufacturing. For example, balloon expandable or self-expandable vascular implants 300 having a structure described in the embodiments above may be laser cut from raw material tubing. In an embodiment, raw Nitinol tubing with an outer diameter of 0.081-inch and a wall thickness of 0.004-inch may be used. The patterns illustrated in the figures may be an “as-cut” configuration of the vascular implant formed from self-expandable material, and thus, may correspond to an expanded state in which the undulation struts are slanted. Alternatively, the patterns illustrated in the figures may be in an expanded configuration of the vascular implant formed from expandable material, and thus, the “as-cut” configuration may instead include longitudinally oriented struts rather than slanted struts.

Laser cutting may be followed by a combination of cleaning, polishing, and passivation processes. For example, in the case of balloon expandable vascular implants 300, the vascular implant 300 may be etched, passivated, and/or electropolished to achieve a surface finish that is clean, atraumatic to vessel tissue, and corrosion resistant. In the case of self-expandable vascular implants 300, the vascular implant 300 may be sand-blasted, etched, electropolished, and passivated to achieve a suitable surface finish.

In addition to finishing the surface of vascular implant 300, various steps may be followed to modify a configuration of vascular implant 300. For example, various heat treatment steps may be applied to a self-expandable vascular implant 300 in order to provide a heat set material memory in the fully expanded configuration. Heat setting may involve expanding vascular implant 300 to the desired configuration using a sequence of heat treating steps. For example, vascular implant 300 may be placed over a mandrel of a desired diameter in each step to sequentially increase the diameter to a deployment diameter, e.g., about 4.25 mm.

Vascular implant 300 may be loaded onto or into a delivery system in numerous manners. For example, in the case of a balloon-expandable implant, a crimping process may reduce the diameter of a laser cut vascular implant 300 to affix the implant struts to a non-compliant or semi-compliant balloon of a balloon delivery catheter. In the case of a self-expandable vascular implant 300, one or more crimping processes may be applied to reduce the diameter of vascular implant 300, e.g., to 0.025 inch, such that vascular implant 300 can be loaded into a delivery sheath of a self-expandable delivery system that constrains vascular implant 300 during delivery. The crimping process may change the orientation of undulation struts from a diagonal orientation in the “as-cut” configuration to a longitudinally oriented configuration in the crimped or unexpanded state. One skilled in the art will appreciate that such crimping and loading processes may apply longitudinal compression to vascular implant 300, and thus, the helix connector schemes described above may be used to provide sufficient axial stiffness to resist buckling of the end of vascular implant 300 during crimping and/or loading.

These and other processes may be performed in accordance with skill in the art. For example, coating processes may be used to coat the implant surface with therapeutic agents, including drugs that have been used in the field of drug-eluting stents, e.g., paclitaxel, zotarolimus, everolimus, sirolimus, etc. These agents may be used alone or in combination with polymer carriers, such as biostable or biodegradable polymers that may be loaded to retain and time-release a therapeutic agent. Thus, the manufacturing processes provided above are illustrative and not limiting of the range of manufacturing processes that may be used to form vascular implant 300 and to prepare the implant for delivery to an aneurysm location within a patient.

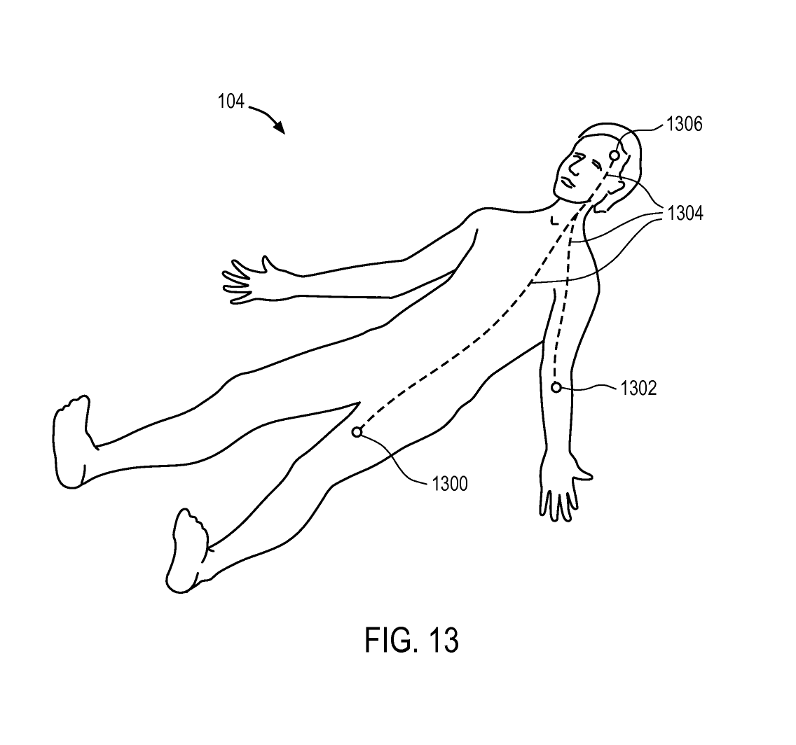

Referring to FIG. 13, a pictorial view of an intravascular access path to an aneurysm site in a patient is shown. An aneurysm in a patient vessel may be accessed through various locations, including a femoral access site 1300 or a radial access site 1302. For example, an intravascular path 1304 may be accessed through those locations using an introducer kit and a guidewire, as is well known. Intravascular path 1304 may then be followed using the guidewire until an aneurysm site 1306 is reached. For example, aneurysm site 1306 may be accessed in a cerebral vessel by a guidewire tracked from femoral access site 1300 through a femoral artery, aorta, carotid artery, and various cerebral vessels of intravascular path 1304.

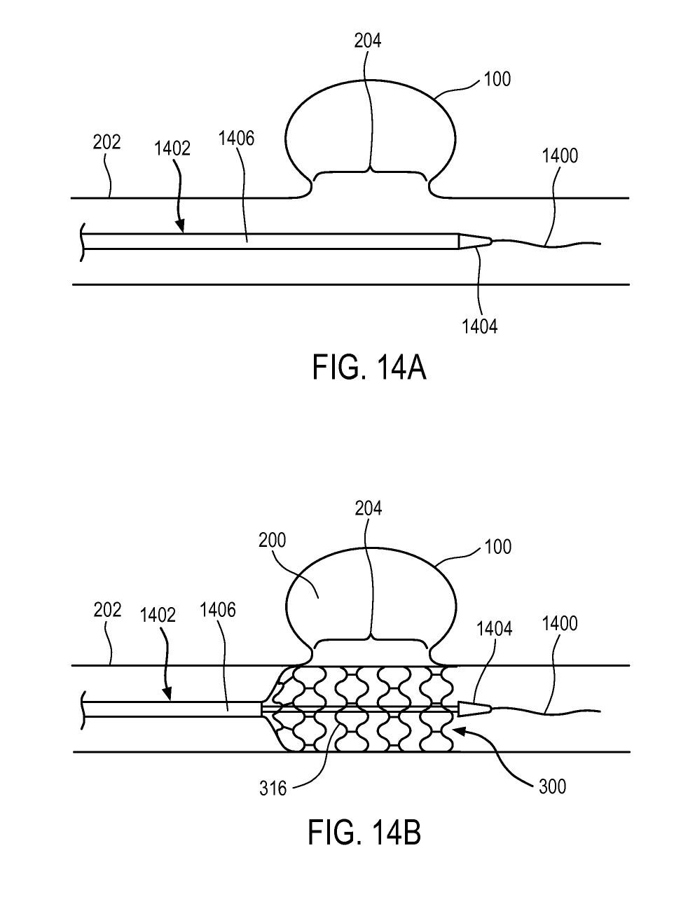

Referring to FIG. 14A, a pictorial view of a delivery system being tracked to an aneurysm site is shown in accordance with an embodiment of the invention. One skilled in the art will recognize that the system illustrated in FIG. 14A may include a construction similar to other delivery systems 1402 used for delivering a self-expandable stent into a patient vasculature. Vascular implant 300 may, however, be a balloon-expandable implant, and thus, delivery and deployment may also be achieved using balloon expandable delivery systems, as is known in the art. After an aneurysm site is accessed by a guidewire 1400, a delivery system 1402 may be delivered over guidewire 1400 until a distal tip 1404 of delivery system 1402 is in the vicinity of the aneurysm site, e.g., distal to aneurysm opening 204. Delivery system 1402 may include outer sheath 1406 to constrain a crimped vascular implant 300 to a delivery diameter. Longitudinal placement of delivery system 1402 may be visualized and controlled according to end markers 410, and/or radiopaque markers associated with delivery system 1402, which may be viewed under fluoroscopy.

Referring to FIG. 14B, a pictorial view of a vascular implant partially deployed from a delivery system at an aneurysm site is shown in accordance with an embodiment of the invention. After vascular implant 300 is positioned relative to aneurysm 100, vascular implant 300 may be deployed into parent vessel 202. In the case of a self-expandable vascular implant 300, outer sheath 1406 may be retracted from distal tip 1404. Thus, vascular implant 300 may expand within parent vessel 202 against aneurysm opening 204. In an embodiment, vascular implant 300 may remain flush with aneurysm opening 204, i.e., vascular implant 300 may assume a cylindrical contour. In an alternative embodiment, vascular implant 300 may protrude slightly into aneurysm sac 200, i.e., a portion of vascular implant 300 covering aneurysm opening 204 may bulge slightly into aneurysm sac 200. In a case of a balloon expandable vascular implant 300, expansion from an unexpanded state to an expanded state may be facilitated by introducing an inflation fluid into a balloon that applies an outward force on vascular implant 300 to cause vascular implant 300 to increase in diameter until it expands against parent vessel 202 to cover aneurysm opening 204. A fully deployed vascular implant 300 may appose parent vessel 202 on either side of aneurysm opening 204 to form a tight seal with parent vessel 202. Thus, blood flow into aneurysm opening 204 may be restricted to blood passing through medial undulations 316 covering aneurysm opening 204, and aneurysm sac 200 may be depressurized. In some embodiments, a pattern density of vascular implant 300 may be altered depending on the intended use. For example, when vascular implant 300 is to be used as a neurovascular stent, a pattern density as shown in FIGS. 3 and 9 may be used. Alternatively, when vascular implant 300 is to be used as a flow diversion device, a pattern density as shown in FIGS. 10-12 may be used. More particularly, one skilled in the art informed by the description above would be able to modify a pattern of vascular implant 300 to achieve an intended design purpose.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A vascular implant having an unexpanded state and an expanded state, the vascular implant comprising:
a plurality of helical scaffolds winding around a longitudinal axis from a first end to a second end of the vascular implant, wherein each helical scaffold includes a terminal undulation at the first end and a plurality of medial undulations helically offset from the terminal undulation between the first end and the second end, wherein each medial undulation includes a short strut connected to a long strut by a medial joint, and wherein the short strut is at least 20% shorter than the long strut.

2. The vascular implant of claim 1, wherein a helical gap winds around the longitudinal axis between a first helical scaffold and a second helical scaffold, and wherein a longitudinal distance across the helical gap is less than a difference in length between the short strut and the long strut.

3. The vascular implant of claim 2, wherein the plurality of helical scaffolds includes three or more helical scaffolds.

4. The vascular implant of claim 3, wherein the helical scaffolds extend along respective slant lines around the longitudinal axis, and wherein the slant lines form a slant angle of less than 45 degrees with a longitudinal line parallel to the longitudinal axis.

5. The vascular implant of claim 2, wherein the first helical scaffold includes a first terminal undulation having a first terminal strut connected to a second terminal strut by a first terminal joint, and wherein the first terminal strut extends across the helical gap and connects directly to the second helical scaffold.

6. The vascular implant of claim 5, wherein the second terminal strut is connected to the second helical scaffold by a helix connector extending across the helical gap.

7. The vascular implant of claim 6, wherein a second helical gap winds around the longitudinal axis between the second helical scaffold and a third helical scaffold, wherein a first long strut of a first medial undulation helically adjacent to the first terminal undulation is connected to the second helical scaffold, and wherein a second long strut of a second medial undulation helically adjacent to a second terminal undulation of the second helical scaffold is not connected to the third helical scaffold.

8. The vascular implant of claim 7, wherein the second terminal undulation includes a third terminal strut connected to a fourth terminal strut by a second terminal joint, wherein the second terminal joint is circumferentially aligned with the first terminal joint, wherein the third terminal strut connects directly to the third helical scaffold across the second helical gap, and wherein a length of the first terminal strut is different than a length of the third terminal strut.

9. The vascular implant of claim 2, wherein the first helical scaffold includes a helix length from the first end to the second end, wherein the first helical scaffold is connected to the second helical scaffold by a plurality of helix connectors extending across the helical gap, and wherein the helix connectors are helically offset from each other by five or more medial undulations of the second helical scaffold over a majority of the helix length.

10. The vascular implant of claim 9, wherein the second helical scaffold includes a second helix length from the first end to the second end, wherein a second helical gap winds around the longitudinal axis between the second helical scaffold and a third helical scaffold, wherein the second helical scaffold is connected to the third helical scaffold by a plurality of second helix connectors extending across the second helical gap, and wherein the second helix connectors are helically offset from each other by three or less medial undulations of the third helical scaffold over a majority of the second helix length.

11. A vascular implant having an unexpanded state and an expanded state, the vascular implant comprising:
a first helical scaffold winding around a longitudinal axis from a first end to a second end of the vascular implant, the first helical scaffold having a first terminal undulation at the first end and a plurality of medial undulations helically offset from the first terminal undulation between the first end and the second end, wherein each medial undulation includes a short strut connected to a long strut by a medial joint; and
a second helical scaffold winding around the longitudinal axis from the first end to the second end, wherein a helical gap extends between the first helical scaffold and the second helical scaffold, and wherein a longitudinal distance across the helical gap is less than a difference in length between the short strut and the long strut.

12. The vascular implant of claim 11, wherein the short strut is at least 20% shorter than the long strut.

13. The vascular implant of claim 12, wherein the helical scaffolds extend along respective slant lines around the longitudinal axis, and wherein the slant lines form a slant angle of less than 45 degrees with a longitudinal line parallel to the longitudinal axis.

14. The vascular implant of claim 12, wherein the first terminal undulation includes a first terminal strut connected to a second terminal strut by a first terminal joint, and wherein the first terminal strut extends across the helical gap and connects directly to the second helical scaffold.

15. The vascular implant of claim 14, wherein the second terminal strut is connected to the second helical scaffold by a helix connector extending across the helical gap.

16. The vascular implant of claim 15, wherein a third helical scaffold winds around the longitudinal axis and is separated from the second helical scaffold by a second helical gap, wherein a first long strut of a first medial undulation helically adjacent to the first terminal undulation is connected to the second helical scaffold, and wherein a second long strut of a second medial undulation helically adjacent to a second terminal undulation of the second helical scaffold is not connected to the third helical scaffold.

17. The vascular implant of claim 16, wherein the second terminal undulation includes a third terminal strut connected to a fourth terminal strut by a second terminal joint, wherein the third terminal strut connects directly to the third helical scaffold across the second helical gap, and wherein a length of the first terminal strut is different than a length of the third terminal strut.

18. The vascular implant of claim 17, wherein the second terminal joint is circumferentially aligned with the first terminal joint.

19. The vascular implant of claim 12, wherein the first helical scaffold includes a helix length from the first end to the second end, wherein the first helical scaffold is connected to the second helical scaffold by a plurality of helix connectors extending across the helical gap, and wherein the helix connectors are helically offset from each other by five or more medial undulations of the second helical scaffold over a majority of the helix length.

20. The vascular implant of claim 19, wherein the second helical scaffold includes a second helix length from the first end to the second end, wherein a third helical scaffold winds around the longitudinal axis and is separated from the second helical scaffold by a second helical gap, wherein the second helical scaffold is connected to the third helical scaffold by a plurality of second helix connectors extending across the second helical gap, and wherein the second helix connectors are helically offset from each other by three or less medial undulations of the third helical scaffold over a majority of the second helix length.
